SEQUENCE LISTING (10 sequences)

SEQ ID NO: 1 - DNA, 38 bp, Artificial Sequence
  Description of Artificial SequenceRibozyme InventorFukuda, Noboru; Ueno, Takahiro; Fukushima, Akiko; Kuroda, Kazumichi.
aacagcuucugaugaguccuugcggacgaaacaaaugu

SEQ ID NO: 2 - DNA, 17 bp, Unknown Organism
  Description of Unknown OrganismT7polymerase
taatacgactcactata

SEQ ID NO: 3 - DNA, 42 bp, Unknown Organism
  Description of Unknown OrganismT7 polymerase
nnnnnnnnnnnnnnnnnnnnnnncctatagtgagtcgtatta

SEQ ID NO: 4 - DNA, 90 bp, Coronavirus
aagcgagcatacattggcggaaacagcttgacaaatgttaaacacagaattagcaaaagcagtggttgcatccccactactagtgccacc

SEQ ID NO: 5 - DNA, 107 bp, MHV RNAvirus
taatacgactcactataggtggcactagtagtggggatgcaaccactgcttttgctaattctgtgtttaacatttgtcaagctgtttccgccaatgtatgctcgctt

SEQ ID NO: 6 - DNA, 31 bp, Artificial Sequence
  Description of Artificial SequencePrimer Inventorfukuda, Noboru; Ueno, Takahiro; Fukushima, Akiko; Kuroda, Kazumichi.
tctgagtaggtacctggcactagtagtgggg

SEQ ID NO: 7 - DNA, 32 bp, Artificial Sequence
  Description of Artificial SequencePrimer Inventorfukuda, Noboru; Ueno, Takahiro; Fukushima, Akiko; Kuroda, Kazumichi
cagtcgtatctagaaagcgagcatacattggc

SEQ ID NO: 8 - DNA, 100 bp, SARS-CoV RNA virus
catcatccggtgatgctacaactgcttatgctaatagtgtctttaacatttgtcaagctgttacagccaatgtaaatgcacttctttcaactgatggtaa

SEQ ID NO: 9 - DNA, 20 bp, Artificial Sequence
  Description of Artificial SequencePrimer Inventorfukuda, Noboru; Ueno, Takahiro; Fukushima, Akiko; Kuroda, Kazumichi
tcatccggtgatgctacaac

SEQ ID NO: 10 - DNA, 22 bp, Artificial Sequence
  Description of Artificial SequencePrimer Inventorfukuda, Noboru; Ueno, Takahiro; Fukushima, Akiko; Kuroda, Kazumichi
ccatcagttgaaagaagtgcat

(19) United States
(12) Patent Application Publication
(10) Pub. No.: US 2010/0273997 A1
(43) Pub. Date: Oct. 28, 2010

(54) Ribozyme to cleave coronavirus gene

(76) Inventors: Noboru Fukuda, Tokyo (JP); Takahiro Ueno, Tokyo (JP); Akiko Fukushima, Tokyo (JP); Kazumichi Kuroda, Tokyo (JP)

Correspondence Address: YOUNG & THOMPSON, 209 Madison Street, Suite 500, Alexandria, VA, 22314, US

(21) Appl. No.: 11/501,049

(22) Filed: Aug. 9, 2006

(30) Foreign Application Priority Data
Aug. 9, 2005 (JP) 2005-231051

(51) Int. Cl. C07H 21/02

(52) U.S. Cl. 536/24.5

(57) ABSTRACT

Provided is a ribozyme to cleave a coronavirus gene and a therapeutic agent for a coronavirus infectious disease. A common base sequence in coronaviruses such as SARS-CoV and MHV was searched to design a ribozyme including a base sequence complementary thereto. Moreover, a therapeutic agent for a coronavirus infectious disease including such ribozyme was obtained.

### BACKGROUND OF THE INVENTION

1. Field of the Invention

The present invention relates to a ribozyme to cleave a coronavirus gene. The present invention further relates to a therapeutic agent for a coronavirus infectious disease that includes the ribozyme.

2. Description of the Related Art

During a period between November 2002 and February 2003, 305 cases including 5 deaths of atypical pneumonia, which is an emergent infectious disease, have been reported in Guangdong Province, China. Such the atypical pneumonia is referred to as “severe acute respiratory syndrome (hereinafter abbreviated as SARS)”, which shakes not only SARS outbreak countries but also the entire world because of high mortality and rapid spread of the infection. On March 25, CDC (United States) discovered a novel virus belonging to a coronavirus family, and on April 12, a Canadian study group has identified an entire sequence of a coronavirus gene. Then, on April 16, WHO has determined that the virus is a causative virus of SARS. The SARS virus is one of coronaviruses and causes zoonotic infection from animals to humans.

After a lapse of about 8 months, a SARS outbreak was over on Jul. 15, 2003, and more than 8,400 cases and about 800 deaths in 30 countries or more around the world were reported to WHO. Another large outbreak has not occurred since the outbreak was over in July, but there is a fear of the SARS outbreak in the future.

It has been clarified that a causative virus of SARS is a novel coronavirus different from the known coronaviruses (SARS coronavirus, hereinafter abbreviated as SARS-CoV), and the sequence of the full length of the genome (about 30,000 bases) has been determined. It hasn't been long since SARS-CoV was discovered, so that the diverse aspects thereof have not been revealed. However, conceivably, there are many virological similarities with other coronaviruses. The viruses belonging to the genus Coronavirus are divided into three groups based on the antigenic cross-reactivities (e.g., homology of the base sequences and amino acid sequences) as shown in FIG. 1. The homology of the viruses belonging to the same group is significantly high compared with that of viruses belonging to different groups, but SARS-CoV has low homology to all viruses belonging to groups 1 to 3 and is considered to belong the fourth group, although some researchers classify SARS-CoV as group 2.

As a therapeutic agent for SARS, various countries are developing vaccines or the like now. In addition, there are also developing a decoction including scutellaria root, gardenia, honeysuckle flower, weeping forsythia, dried rehmannia root, gypsum, flos chrysanthemi indici, basket fern, balloonflower root, mint, arctium fruit, and liquorice (See, for example, JP 2005-2084A) and the like, but there has been no report of a therapeutic agent effective against SARS virus. Meanwhile, in recent years, as a therapeutic method for a viral disease such as SARS, a gene therapy using a ribozyme has attracted attention.

A ribozyme is RNA having an RNA-cleaving activity and has a hairpin or hammerhead structure, or the like, as shown in FIG. 2. Stem I and Stem III, which have base sequences complementary to that of a target mRNA, are linked to the target mRNA, and a subsequent sequence to the GUC sequence of the target mRNA is cleaved by the enzyme activity to cleave RNA of a conserved sequence. A ribozyme has high specificity to a target gene, and once a ribozyme cleaves mRNA, it detaches from the mRNA and hybridizes to another mRNA to repeat cleavage. Many study groups are now developing a ribozyme that has high specificity to a target gene and has such characteristics for human immunodeficiency virus (HIV) or the like (See, for example, JP 2003-507037 A).

However, a ribozyme having such characteristics for coronaviruses such as SARS virus has not been obtained. As coronaviruses other than SARS virus, the following viruses are known: human coronavirus which causes cold symptoms in a human; porcine transmissible gastroenteritis virus which causes gastroenteritis in a pig; dog coronavirus which causes diarrhea symptoms in a dog; mouse hepatitis virus which causes hepatitic and diarrhea symptoms in a mouse; and the like, but ribozymes for such viruses have not been obtained yet. Therefore, it is desirable to develop a ribozyme effective in a therapy for a coronavirus infectious disease such as SARS.

### SUMMARY OF THE INVENTION

An object of the present invention is to provide a ribozyme to cleave a loop structure of a coronavirus gene. A further object of the present invention is to provide a therapeutic agent for a coronavirus infectious disease that includes the ribozyme.

The inventors of the present invention have made extensive studies to solve the above-described problems, and as a result they have identified a base sequence common to coronaviruses such as SARS-CoV and mouse hepatitis virus (hereinafter abbreviated as MHV) to develop a ribozyme for a coronavirus infectious disease such as SARS and designed a ribozyme including a base sequence complementary thereto to create a ribozyme to cleave loop structures of these coronaviruses, thereby completing the present invention. In addition, they have completed a therapeutic agent for a coronavirus infectious disease that includes the ribozyme.

That is, the present invention relates to the following (1) to (9): ribozymes to cleave a coronavirus gene and a therapeutic agent for a coronavirus infectious disease.

(1) A ribozyme, which cleaves a gene of a virus belonging to the coronavirus family.

(2) A ribozyme according to the above item (1), which recognizes and cleaves a specific base sequence of a gene of a virus belonging the coronavirus family.

(3) A ribozyme according to the above item (2), in which the specific base sequence is a sequence including GUC.

(4) A ribozyme according to the above item (2) or (3), in which the specific base sequence is located in a loop structure.

(5) A ribozyme having a sequence of SEQ ID NO: 1 in SEQUENCE LISTING.

(6) A ribozyme according to any one of the above items (1) to (5), which is an RNA/DNA chimeric ribozyme.

(7) A method of designing a ribozyme, which includes selecting a specific base sequence of a coronavirus, in which the ribozyme includes a sequence complementary thereto.

(8) A method of designing a ribozyme, which includes selecting a specific base sequence by combining at least two of the following steps 1) to 3):

1) selecting a base sequence in a conserved region of a coronavirus gene; 2) selecting a base sequence in a common region of a coronavirus gene;

3) selecting a base sequence that includes a loop structure of a coronavirus gene.

(9) A therapeutic agent for a coronavirus infectious disease, which includes a ribozyme according to any one of the above items (1) to (6).

Use of a ribozyme to cleave a coronavirus gene that has been established by the present invention and a therapeutic agent for a coronavirus infectious disease that includes the ribozyme enables an effective therapy for a coronavirus infectious disease such as SARS.

### BRIEF DESCRIPTION OF THE DRAWINGS

In the accompanying drawings:

FIG. 1 shows an evolutionary tree of coronaviruses;

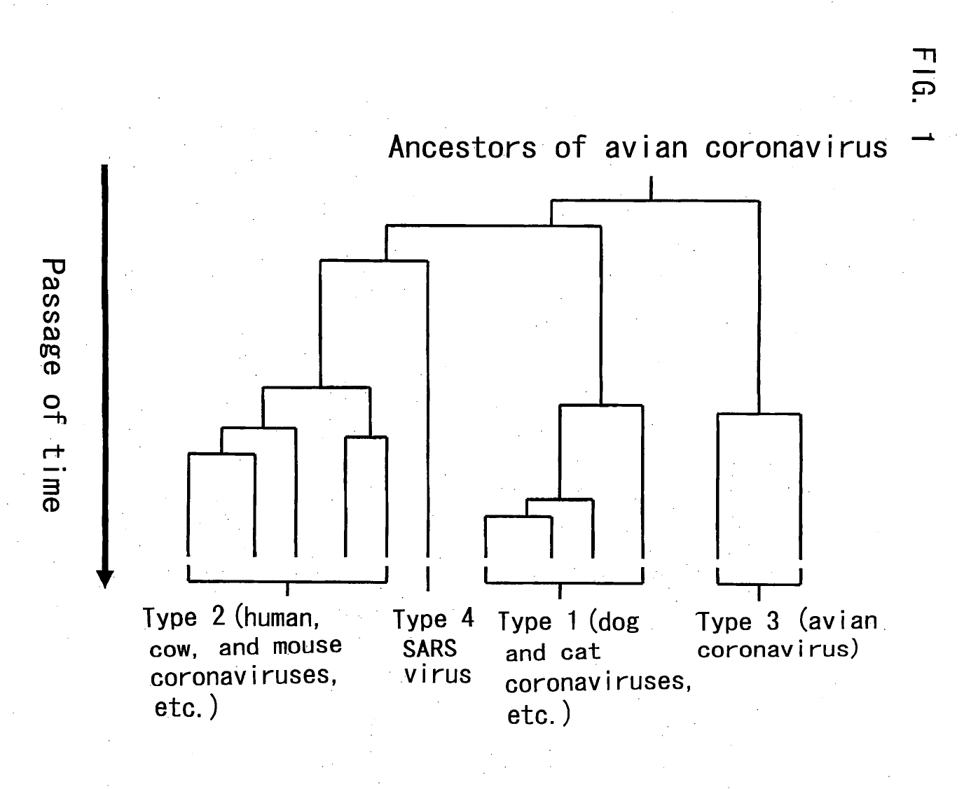

FIG. 2 shows a basic structure of a ribozyme;

FIG. 3 shows a structure of SARS-CoV gene;

FIG. 4 shows a secondary structure of RNA at a common site on MHV and SARS-CoV genes (Example 1);

FIG. 5 shows ribozyme and mismatch ribozyme against a coronavirus (Example 1);

FIG. 6 shows results of confirmation of cleavage of MHV RNA by a ribozyme (Example 2);

FIG. 7 shows details of pcDNA 3.1 (Example 3);

FIG. 8 shows effects of a ribozyme on mRNA of MHV (Example

FIG. 9 shows effects of a ribozyme on mRNA of MHV (Example 3);

FIG. 10 is a graph showing the results of suppression of MHV expression by a ribozyme (Example 4); and

FIG. 11 shows results of confirmation of cleavage of SARS-CoV RNA by a ribozyme (Example 5).

### DETAILED DESCRIPTION OF THE INVENTION

In the case of a ribozyme of the present invention, a target mRNA to be cleaved is mRNA of a coronavirus. Examples thereof include mRNA of SARS-CoV that causes a severe disease (SARS) in human and mRNA of MHV.

In addition, viruses other than coronaviruses may also be targets of the present invention as long as the ribozyme of the present invention recognizes and cleaves the genes and suppresses the expressions, and the viruses are not limited to coronaviruses. For example, in the case of designing a ribozyme based on a base sequence of a conserved region or a common region in a coronavirus gene, other genes may also be targets of the present invention as long as the base sequence in a conserved region or a common region recognizes and cleaves the genes and suppresses the expressions.

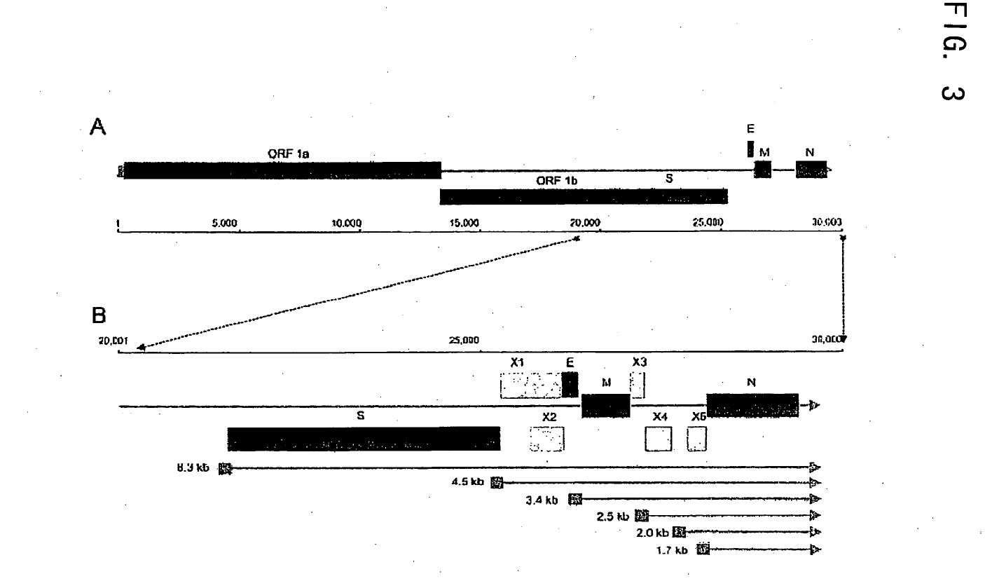

A coronavirus including SARS-CoV is an enveloped virus with characteristic spikes (about 20 nm), and a coronavirus particle is in the circle, ellipse, or polymorphic form with a diameter of about 100 to 200 nm. The spikes on the surface of the particle are composed of S-protein. Meanwhile, an envelope further includes membrane (M) and envelope (E) proteins. The genomic RNA is surrounded by the envelope, and a nuclear (N) protein is linked thereto to form a helical nucleocapsid. A coronavirus including SARS-CoV has (+)-strand genomic RNA with about 30 kb, which is the largest size of known viral RNAs. The genomic RNA has a cap structure at the 5′-end and has poly (A) at the 3′-end. The genome with 30 kb has a leader sequence with about 70 bases at the 5′-end, and in the downstream region, it has RNA polymerases (open reading flames: ORFs 1a and 1b), S, E, M, and N in this order. Among them, as shown in FIG. 3, SARS-CoV has several ORFs (X3, X4, and X5) between M and N gene, which are not present in the genes of other coronaviruses.

In general, coronaviruses have high species specificity and do not infect animals other than a definitive host, but SARS-CoV infects not only human but also monkey, cat, ferret (References 1 and 2), mouse, rat, and the like. The high pathogenicity of SARS-CoV is probably attributable to a host reaction induced by the virus rather than direct cell damage due to viral proliferation (References 3 and 4). Conceivably, pneumonia caused by infection with SARS-CoV causes symptoms similar to a severe inflammatory reaction due to cytokines, as is the case for influenza H5N1 infection developed in 1997, and results from induction of an abnormally severe inflammatory reaction in a host attributable to a large amount of produced cytokines (cytokine storm).

Reference 1: Fouchier RAM, Kuiken T, Schutten M et al. Koch's postulates fulfilled for SARS virus. Nature 423: 240-, 2003.

Reference 2: Martina BEE, Haagmans B L, Kuiken T et al. SARS virus infection of cats and ferrets. Nature 425: 915, 2003.

Reference 3: Nicholls J M, Poon L M, Lee K C et al. Lung pathology of fatal severe acute respiratory syndrome. Lancet 361: 1773-1778, 2003.

Reference 4: Peiris J S M, Chu C M, Cheng V C C et al. (2003) Clinical progression and viral load in a community outbreak of coronavirus-associated SARS pneumonia; a prospective study. Lancet. 361: 1767-1772, 2003.

Meanwhile, MHV is an RNA virus that belongs to the coronavirus family and has an envelope, and the virus particle has a spike-like structure on its outside surface. This virus includes many known isolates having slightly different properties such as pathogenicity and antigenecity. Examples thereof include A-59, JHM, MHV-2, S, and nu-67. These viral strains proliferate in various established cells such as DBT and NCTC-1469, and in the proliferation, many strains exhibit characteristic cytopathic effects such as formation of syncytiotrophoblastic giant cells.

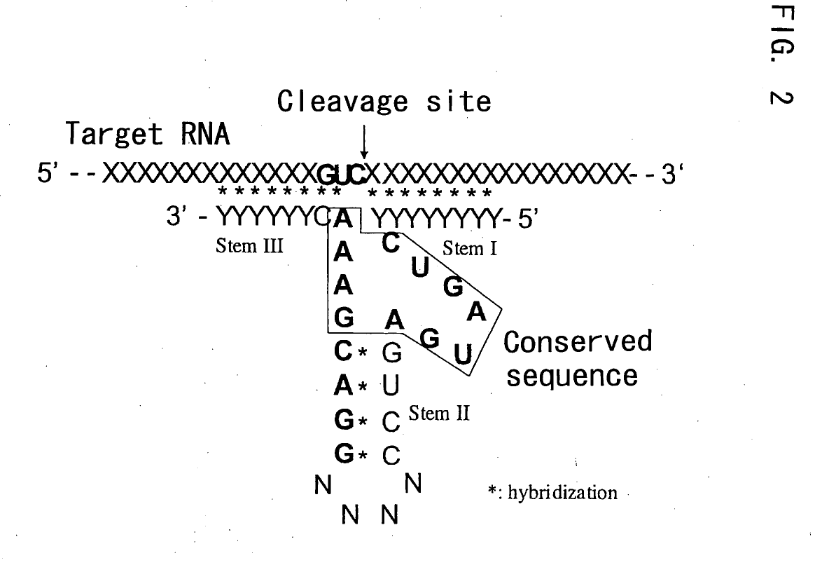

The ribozyme of the present invention preferably recognizes a specific base sequence in a target mRNA to be cleaved. For example, as shown in FIG. 2, the ribozyme preferably has base sequences for linking a target mRNA (Stem I and Stem III), which are complementary to the sequence of the target mRNA, and a conserved sequence for cleaving the target mRNA, which has an enzyme activity to cleave RNA.

Based on these base sequences, the ribozyme can be specifically linked to the target mRNA to be cleaved and cleave the target mRNA by the enzyme activity to cleave RNA of the conserved sequence.

The base sequence to be recognized to cleave a target mRNA is preferably a GUC sequence. Preferably, the ribozyme of the present invention recognizes the GUC sequence and cleaves the subsequent base sequence in mRNA.

The base sequence for linking a ribozyme to a target mRNA is a sequence complementary to a specific sequence in the target mRNA, and a sequence in which some bases are deleted, substituted, or inserted may also be used as long as the ribozyme sufficiently recognizes and is linked to the base sequence.

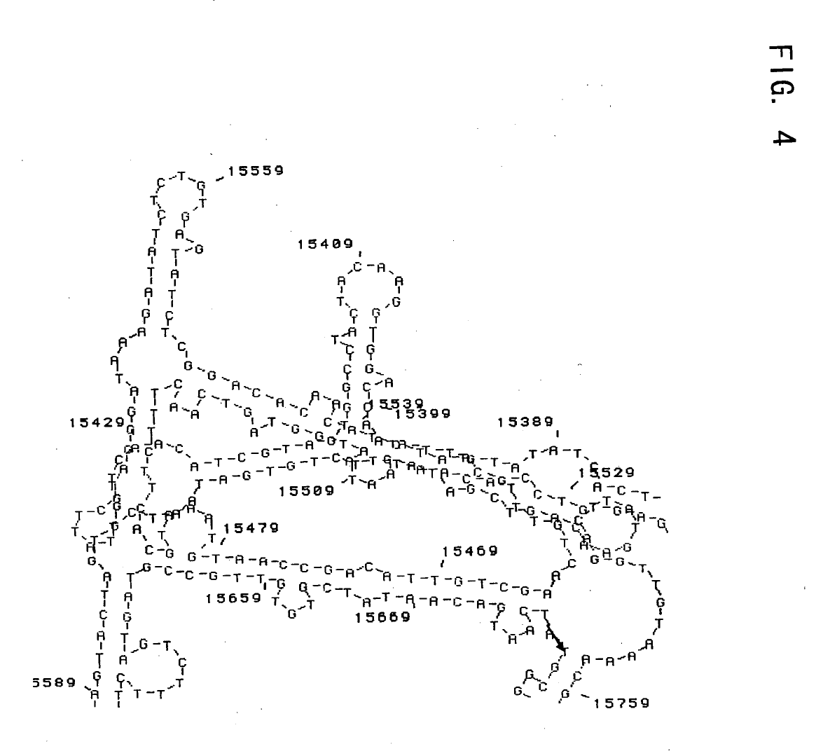

Moreover, the base sequence in a target mRNA to be cleaved by the ribozyme of the present invention is preferably a moiety of a loop structure. The term “loop structure” in the present invention refers to a moiety having a loop-like structure in the case where the target mRNA has a secondary structure as shown in FIG. 4. Note that a moiety having a structure other than the loop-like structure is referred to as “stem structure”.

A base sequence to be cleaved is preferably a moiety of a loop structure. Because in the case where a target mRNA is cleaved by a ribozyme, the tertiary structure of the resultant target mRNA in which the loop structure has been cleaved is drastically changed, resulting in suppression of the functions of the target mRNA, while the tertiary structure of the resultant target mRNA in which the stem structure has been cleaved is not drastically changed, resulting in no suppression of the functions of the target mRNA, in some cases.

The full length of the ribozyme of the present invention is not particularly limited but is 30 bases or more, preferably 35 to 40 bases.

In designing the ribozyme of the present invention, in the case of designing a ribozyme to cleave a loop structure of a coronavirus, base sequences of some kinds of viruses belonging to the coronavirus family may be used to select and design a specific base sequence by combining at least two of the following selecting method 1) to 3):

1) Selecting a base sequence in a conserved region of a coronavirus gene;

2) Selecting a base sequence in a common region of a coronavirus gene; and

3) Selecting a base sequence including a loop structure in a coronavirus gene.

Furthermore, as shown in FIG. 2, it is preferable to design the ribozyme of the present invention so that the base sequences complementary to those of a target mRNA (Stem I and Stem III) are the same or have different lengths in which only a few bases are different, and the ribozyme has Stem II including a conserved sequence at the center position.

For example, a ribozyme to recognize and cleave SARS-CoV gene may be designed by analyzing the secondary structure of a base sequence of RNA in a conserved region of SARS-CoV gene as a coronavirus gene with a computer to search for a loop structure including a GUC sequence. Meanwhile, a ribozyme to recognize and cleave SARS-CoV and MHV genes may be designed by identifying a common base sequence from base sequences in the respective conserved regions of SARS-CoV and MHV genes and analyzing the secondary structure with a computer to search for a loop structure including a GUC sequence. The designing method may also be used for genes of viruses other than coronaviruses.

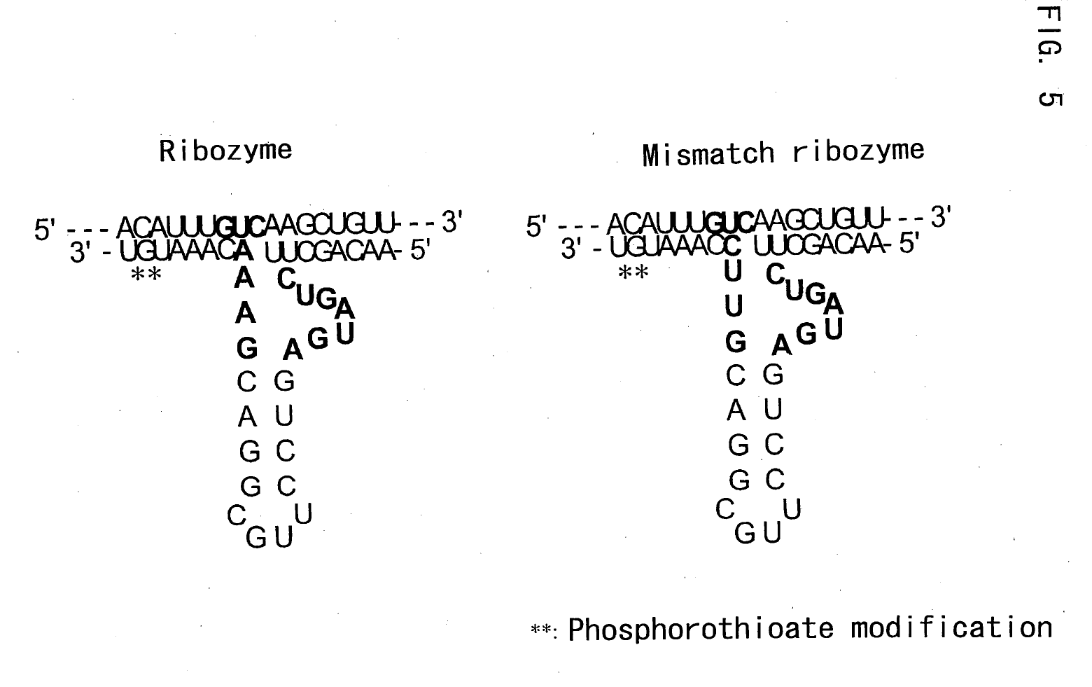

Meanwhile, the base sequences to be used for linkage selected by the above-described method depend on used target mRNAs, so that the designed ribozyme may specifically recognize and cleave a target mRNA. In general, a ribozyme is RNA but may have an RNA/DNA chimeric structure to prevent decomposition by an RNA cleavage enzyme present in a living body. As shown in FIG. 5, the ribozyme against SARS coronavirus was designed so as to have an RNA structure only in a conserved region and a DNA structure in other regions. In addition, the two bases at the 3′-end side were phosphorothioated by chemical modification so as to become resistant to a ribonuclease and so as not to decompose in administration to a living body.

A preferable ribozyme designed by the present invention is, for example, a ribozyme that may recognize and cleave SARS-CoV and MHV as coronaviruses and suppress the expressions thereof, and examples of the ribozyme to be used include a ribozyme of SEQ ID NO: 1 in SEQUENCE LISTING.

A ribozyme designed by the present invention may be mass-produced by any of the known methods. For example, based on the method of Uhlenbeck (Reference 5), T7 RNA polymerase is added to a template DNA linked to T7 RNA polymerase promoter, to thereby yield a ribozyme RNA. Meanwhile, the ribozyme may also be synthesized using an RNA synthesizer. The potency of the resultant ribozyme may be examined by: confirming whether the ribozyme cleaves part of or entire of virus or gene to be cleaved or not; or confirming whether the ribozyme suppresses the expression of the virus or gene or not.

Reference 5: Uhlenbeck O C. A small catalytic oligoribonucleotide. Nature 328:596-600, 1987.

A therapeutic agent for a coronavirus infectious disease to be used that includes the ribozyme of the present invention is not particularly limited as long as it may be one that includes the ribozyme of the present invention as an active ingredient. The coronavirus infectious disease particularly preferably includes diseases caused by SARS virus or MHV, or the like. The therapeutic agent of the present invention may include a stabilizer or the like in addition to the active ingredient of the ribozyme. The form of the therapeutic agent is not particularly limited, but in the case where SARS virus is targeted, it is particularly preferably an inhalant.

Hereinafter, the present invention will be described in more detail by way of examples, but it is not limited thereto.

### Example 1

### Preparation of Ribozyme

1. Molecular design of ribozyme to MHV and SARS-CoV

A base sequence common to MHV and SARS-CoV genes was identified, and the secondary structure thereof was analyzed by a computer to search for a loop structure including a GUC sequence as shown in FIG. 4. As shown in FIG. 5, a hammerhead ribozyme having a sequence complementary to that of the region was designed, and as a control, a mismatch ribozyme, in which three bases in a conserved sequence having an enzyme activity were substituted, was designed.

### 2. Synthesis of Ribozyme

1) Based on the method of Uhlenbeck, T7 RNA polymerase was added to a template DNA linked to T7 RNA polymerase promoter, to thereby yield ribozyme RNA.

2) A template DNA of T7 RNA was shown below and in SEQ ID NOS: 2 and 3 in SEQUENCE LISTING.

5′-TAATACGACTCACTATA-3′
3′-ATTATGCTGAGTGATATCNNNNNNNNNNNNNNNNNNNNNNN-5′

+1

The base indicated with “+1” is “C” with which a T7 RNA polymerase easily reacts. The sequence from the “+2” base represents a template DNA structure of the ribozyme RNA, and based on the sequence, the ribozyme RNA is synthesized. The actual template DNA structure of the ribozyme RNA having a sequence from the 5′-end toward the 3′-side (to the “+1” base) are shown in SEQ ID NO: 4 in SEQUENCE LISTING. The sequence corresponds to the sequences of the 15648th-15654th bases in MHV gene and the 15454th-15470th bases in SARS virus gene.

3) T7 RNA polymerase and the template DNA of the ribozyme were mixed (3 μg/μl each), and the mixture was heated at 90° C. for 3 minutes and immediately cooled to 4° C.

4) 300 U of T7 polymerase, 5 μl of α-32P-CTP (3,000 Ci/mmol), 50 U of an RNase inhibitor, and 50 μl of a transcription reaction buffer (40 mM Tris-HCl (pH 8.0), 0.5 mM rNTP, 8 mM MgCl₂, 5 mM DTT, and 2 mM spermidine) were added thereto, and the mixture was incubated at 37° C. for 4 hours.

5) 100 μl of phenol and chloroform were added thereto, followed by vortexing.

6) The mixture was centrifuged at 16,000 rpm for 30 seconds, and the supernatant was transferred.

7) Chloroform and isoamyl alcohol were added thereto, followed by vortexing. Then, the mixture was centrifuged at 16,000 rpm for 30 seconds, and the supernatant was transferred.

8) 200 μl of 100% ethanol was added thereto, and the mixture was centrifuged at 16,000 rpm for 15 minutes, followed by discarding the supernatant.

9) The resultant RNA pellet was washed with 75% ethanol twice and dissolved in 5 μl of DEPC water, to thereby synthesize a ribozyme.

### Example 2

### MHV RNA Cleavage Experiment

### 3. Confirmation of Cleavage of Target RNA by Ribozyme

### 1) Synthesis of Target RNA

An oligonucleotide with about 100 bases, which includes a site to cleave a ribozyme in MHV RNA, was used as a target RNA. T7 RNA polymerase was added to a template DNA of the target RNA linked to T7 RNA polymerase promoter, and the same procedures as those for synthesis of a ribozyme were performed to synthesize the target RNA.

2) Cleavage of target RNA by ribozyme

a) A labeled ribozyme (2.4 μM), 1 μl of a mismatch ribozyme (2.4 μM), and the target RNA (240 nM) were mixed in 50 mM Tris HCl (pH 8.0), and the mixture was incubated at 90° C. for 1 minute and allowed to stand for 30 minutes at room temperature.

b) 250 mM MgCl₂ was added thereto.

c) The mixture was incubated at 37° C. for 1, 4, and 8 hours.

d) 10 μl of Blue duce was added thereto, and the mixture was incubated at 90° C. for 2 minutes and cooled.

e) The mixture was electrophoresed in a 6% polyacrylamide gel.

### 3. Results

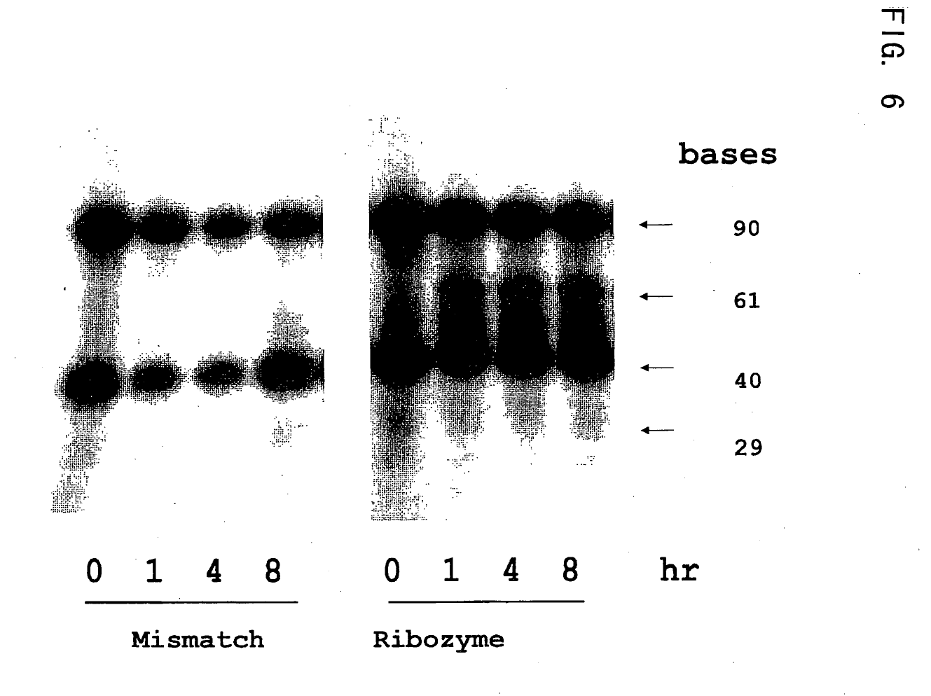

As shown in FIG. 6, in all cases of the incubation times of 1, 4, and 8 hours, the ribozyme (40 bases) cleaved the target RNA (90 bases) in the presence of Mg²⁺ into oligonucleotides with 61 and 29 bases. However, the mismatch ribozyme did not cleave it.

### Example 3

### MHV RNA Expression Suppression Experiment

1) For an oligonucleotide with about 100 bases, which includes a site to cleave a ribozyme in MHV RNA, PCR primers were selected by Primer 3 input (http://frodo.wi.mit.edu/cgi-bin/primer3/primer3_www.cgi). The sequence of an oligonucleotide including the site to cleave it is shown in SEQ ID NO: 5 in SEQUENCE LISTING. Meanwhile, the sequences of the selected primers are shown in SEQ ID NOS: 6 and 7 in SEQUENCE LISTING.

2) A 5′-→3′ oligonucleotide having a sequence corresponding to that of the region between the primers, a complementary 3′-→5′ oligonucleotide, and the primers were synthesized.

3) The 5′-→3′) The oligonucleotide and complementary 3′-→5′ oligonucleotide were annealed, and the resultant product was used as a template to perform PCR.

4) The PCR products were electrophoresed, and a band corresponding to oligonucleotides with 100 bases was cut out and purified.

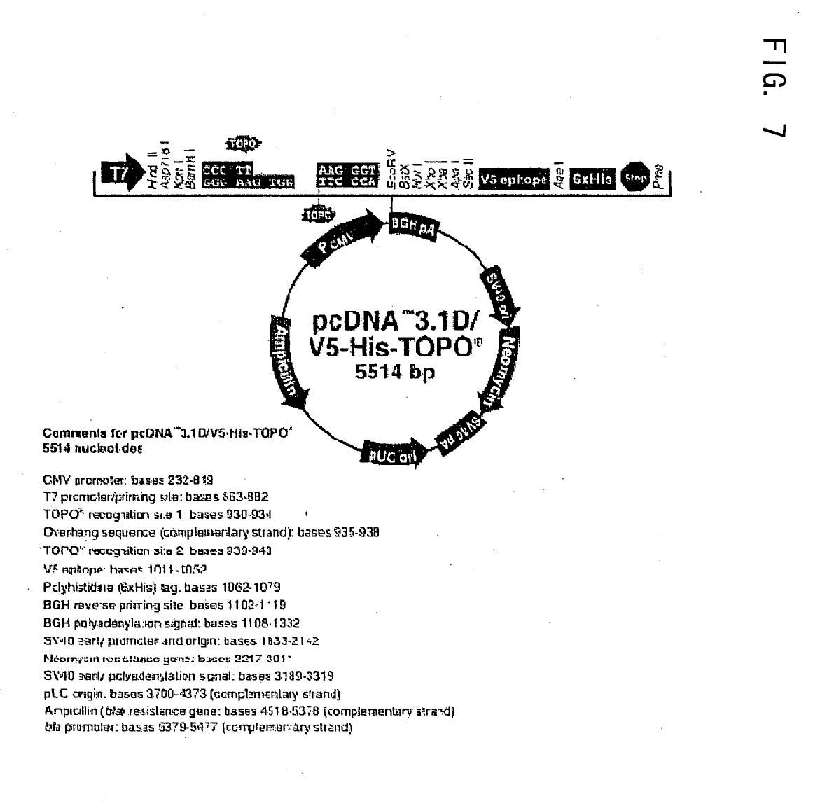

5) In accordance with the manual for pcDNA 3.1, the PCR products were inserted in pcDNA 3.1 (FIG. 7).

6) The resultant plasmid was inserted in competent E. coli.

7) Colonies were confirmed in a medium supplemented with ampicillin.

8) Colony PCR was performed to confirm reconstruction.

9) MHV RNA fragments were confirmed by direct sequence analysis.

10) An MHV RNA expression vector was transfected into 3T3 cells with lipofectin, followed by incubation for 1 hour.

11) The ribozyme and mismatch ribozyme (10⁻⁹, 10⁻⁸, and 10⁻⁷ M) were inserted with lipofectin, followed by incubation for 18 hours.

12) The RNA was extracted with TRIZOL Reagent, and the MTV RNA fragments were subjected to RT-PCR.

13) It was confirmed that the ribozyme suppress the expression of MHV RNA fragment mRNA by electrophoresis and a bioanalyzer.

### Results

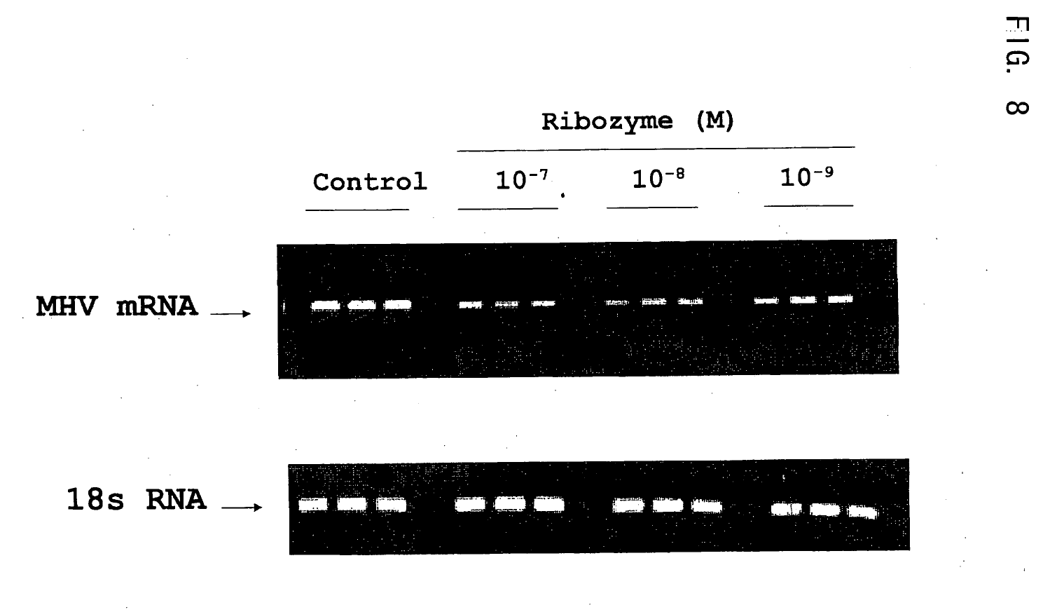

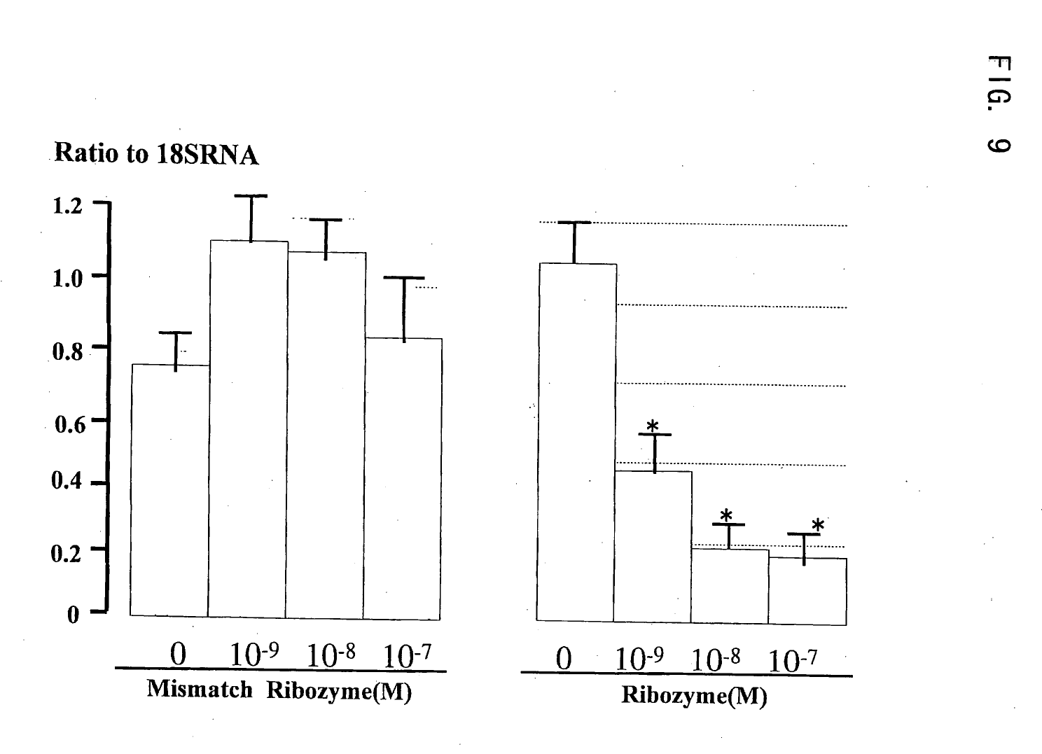

As shown in FIG. 8, the ribozyme significantly suppressed the expression of MHV mRNA, but the mismatch ribozyme did not suppress it. In addition, as shown in FIG. 9, it was revealed that the ribozyme suppressed the expression depending on the concentration.

### Example 4

### MHV Suppression by Ribozyme in DBT Cell

1) DBT cells were prepared in a 24-well plate at 2.8×10⁵/0.5 ml well.

2) 24 hours later, 1.0 μM mismatch ribozyme and ribozyme were separately delivered with a delivery reagent, 20-kDa polyethyleneimine (ExGen 500).

3) 2 hours later, MHV (0.01 MOI) was allowed to infect the cells, and the supernatants were removed after a lapse of 45 minutes. Then, the cells were washed twice, and the culture medium was exchanged for fresh one.

4) 12 hours later, the supernatants were stored.

5) The stored supernatants were inoculated to fresh DBT cells, and the MHV levels in the supernatants were determined based on the number of syncytiotrophoblastic giant cells.

### Results

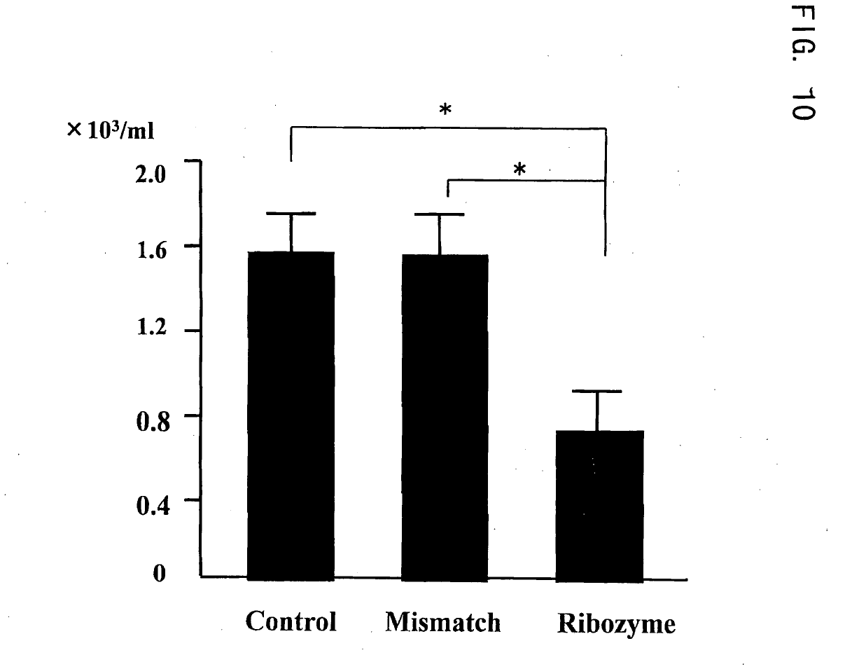

As shown in FIG. 10, there was no significant difference between the control group and mismatch ribozyme group on the numbers of the syncytiotrophoblastic giant cells generated by MHV in the supernatants obtained after 12 hours. However, when compared between the control and ribozyme groups, and between the mismatch ribozyme and ribozyme groups, the cell number in the ribozyme-administered group was significantly suppressed. Note that the transfection efficiency of the FITC-labeled ribozyme into the DBT cells with ExGen 500 was found to be about 60%.

### Example 5

### SARS Virus RNA Cleavage Experiment

1) For an oligonucleotide with about 100 bases, which includes a site to cleave a ribozyme in SARS-CoV RNA, PCR primers were selected by Primer 3 input (http: frodo.wi.mit.edu/cgi-bin/primer3/primer3_www cgi). The sequence of an oligonucleotide including the site to cleave it is shown in SEQ ID NO: 8 in SEQUENCE LISTING. Meanwhile, the sequences of the selected primers are shown in SEQ ID NOS: 9 and 10 in SEQUENCE LISTING.

2) A 5′-→3′ oligonucleotide having a sequence corresponding to that of the region between the primers, a complementary 3′-→5′ oligonucleotide, and the primers were synthesized.

3) The 5′-→3′ oligonucleotide and complementary 3′-→5′ oligonucleotide were annealed, and the resultant product was used as a template to perform PCR.

4) The PCR products were electrophoresed, and a band corresponding to oligonucleotides with 100 bases was cut out and purified.

5) In accordance with the manual for pcDNA 3.1, the PCR products were inserted in pcDNA 3.1.

6) The resultant plasmid was inserted in competent E. coli.

7) Colonies were confirmed in a medium supplemented with ampicillin.

8) Colony PCR was performed to confirm reconstruction.

9) SARS-CoV RNA fragments were confirmed by direct sequence analysis.

10) A SARS-CoV RNA expression vector was cleaved with a restriction enzyme.

11) 30 U of T7 polymerase, 5 μl of α-32P-UTP (800 Ci/mmol), 1 μl of 10 mM ATP, 1 μl of 10 mM CTP, 1 μl of 10 mM GTP, and 9 μl of a transcription reaction buffer (with DTT) were added to 1 μg of a template, and the mixture was incubated at 37° C. for 20 minutes.

12) 9 μl of a ribozyme (100 μM), 9 μl of mismatch ribozyme (100 μM), 1 μl of a labeled target RNA (240 nM), and 1 μl of MgCl₂ (10 mM) were mixed in 50 mM Tris HCl (pH 7.5), and the mixture was incubated at 90° C. for 1 minute and immediately cooled.

13) The mixture was incubated at 37° C. for 2 hours.

14) 10 μl of Blue duce was added thereto, and the mixture was incubated at 90° C. for 2 minutes.

15) The mixture was electrophoresed in a 5% polyacrylamide gel.

### Results

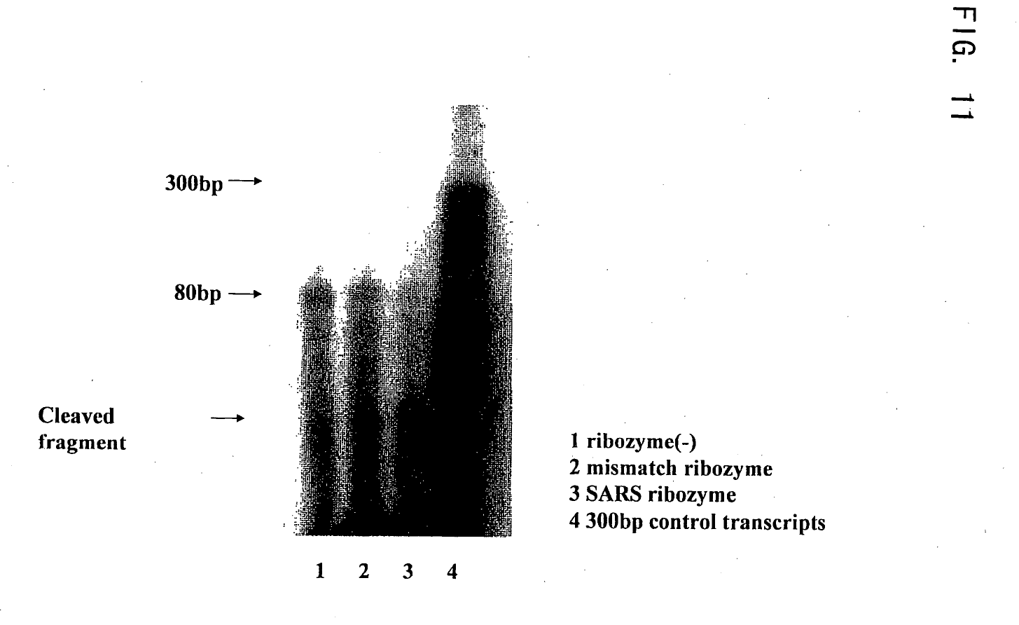

As shown in FIG. 11, the target RNA with 80 bases was not cleaved by the control and mismatched ribozyme, but in the case of the ribozyme-administered group, the target RNA was cleaved and reduced, resulting in appearance of cleaved fragments.

### INDUSTRIAL APPLICABILITY

Use of the ribozyme of the present invention as an active ingredient of a therapeutic agent for a coronavirus infectious disease enables an effective therapy for a coronavirus infectious disease such as SARS.

1. A ribozyme, which cleaves a gene of a virus belonging to the coronavirus family.

2. A ribozyme according to claim 1, which recognizes and cleaves a specific base sequence of a gene of a virus belonging to the coronavirus family.

3. A ribozyme according to claim 2, wherein the specific base sequence is a sequence including GUC.

4. A ribozyme according to claim 2, wherein the specific base sequence is located in a loop structure.

5. A ribozyme having a sequence of SEQ ID NO: 1 in SEQUENCE LISTING.

6. A ribozyme according to claim 1, which is an RNA/DNA chimeric ribozyme.

7. A method of designing a ribozyme, comprising selecting a specific base sequence of a coronavirus, wherein the ribozyme includes a sequence complementary thereto.

8. A method of designing a ribozyme, comprising selecting a specific base sequence by combining at least two of the following steps 1) to 3):
1) selecting a base sequence in a conserved region of a coronavirus gene;
2) selecting a base sequence in a common region of a coronavirus gene;
3) selecting a base sequence that includes a loop structure of a coronavirus gene.

9. A therapeutic agent for a coronavirus infectious disease, comprising the ribozyme according to claim 1.

10. A ribozyme according to claim 3, wherein the specific base sequence is located in a loop structure.
